(19) United States
(12) Patent Application Publication
(10) Pub. No.: US 2019/0083036 A1
(43) Pub. Date: Mar. 21, 2019

(54) BIOSIGNAL MEASURING METHOD AND SYSTEM USING EARPHONE

(71) Applicant: WELT CORP., LTD., Seoul (KR)

(72) Inventors: Seong Ji KANG, Seoul (KR); Hye Kang ROH, Seoul (KR); Hwa Young JEONG, Incheon (KR)

(73) Assignee: WELT CORP., LTD., Seoul (KR)

(21) Appl. No.: 15/833,586

(22) Filed: Dec. 6, 2017

(30) Foreign Application Priority Data
Sep. 20, 2017 (KR) 10-2017-0121379

(51) Int. Cl. A61B 5/00; A61B 5/024; A61B 5/0404; A61B 5/01

(52) U.S. Cl. CPC A61B 5/6817; A61B 5/0002; H04R 1/10; A61B 5/0404; A61B 5/01; A61B 5/02438

(57) ABSTRACT

A biosignal measuring method and system using an earphone are provided, in which the biosignal measuring method includes sensing the earphone being connected to a user terminal, in which the earphone includes a measuring unit for measuring a biosignal, transferring a sensor driving signal from the user terminal to the connected earphone during a predetermined interval, and driving the measurement unit according to the sensor drive signal to measure the biosignal.

### TECHNICAL FIELD

This application claims priority from Korean Patent Application No. 10-2017-0121379, filed on Sep. 20, 2017 in the Korean Intellectual Property Office, the disclosure of which is incorporated herein by reference in its entirety.

The present disclosure relates to a biosignal measuring method and system, and more particularly, to a biosignal measuring method and system using an earphone.

### BACKGROUND ART

In recent years, various services have been provided to support user healthcare using smartphones. For example, a variety of smartphone applications have been developed and commercialized, which can manage users' daily life habits such as exercise and eating habits, sleep patterns, biorhythms and so on. However, smartphone applications that have been commercialized so far are no better than just using the inertial sensor built in the smartphone to monitor the activities of the users, or receiving users' health information directly from the users.

It is important to measure and manage the biosignal such as body temperature to determine the health condition of the user. Accordingly, there is a need to develop a method for naturally measuring and managing biosignals by using the smartphones that users always carry around.

### SUMMARY

Exemplary embodiments of the present inventive concept overcome the above disadvantages and other disadvantages not described above. Also, the present inventive concept is not required to overcome the disadvantages described above, and an exemplary embodiment of the present inventive concept may not overcome any of the problems described above.

It is an object of the present disclosure to provide a biosignal measuring method and system using an earphone.

According to an embodiment of the present disclosure, there is provided a biosignal measuring method to solve the technical problems mentioned above, which may include sensing the earphone being connected to a user terminal, in which the earphone includes a measuring unit for measuring a biosignal, transferring a sensor driving signal from the user terminal to the connected earphone during a predetermined interval, and driving the measurement unit according to the sensor drive signal to measure the biosignal.

The predetermined interval may be a mute interval during which an audio electric signal is not transferred from the user terminal.

The mute interval may be defined as an interval spanning from an end of playing a first content to a start of playing a second content in the user terminal.

The sensor driving signal may be a signal having a frequency outside audible frequency range.

The sensor driving signal may have a voltage equal to or greater than a predetermined intensity.

The sensor driving signal may have a frequency outside a predetermined frequency range.

The predetermined frequency range may be equal to or greater than a first frequency and equal to or less than a second frequency.

The biosignal may be at least one of body temperature, electrocardiogram (ECG), electroencephalogram (EEG), electrocardiogram (EMG), electrooculogram (EOG),) and pulse rate.

The biosignal may be a body temperature, and when a duration in which the earphone is connected to the user terminal is equal to or less than a predetermined reference value, the user terminal may display the body temperature of the user which is predicted based on a rate of change of the temperature measured at the measuring unit.

According to an embodiment of the present disclosure, there is provided a biosignal measuring system using an earphone, which may include an earphone including a measuring unit for measuring a biosignal, and a user terminal configured to transfer a sensor driving signal to drive the measuring unit during a mute interval in which an audio electric signal is not outputted to the earphone, and receive measured biosignal from the measuring unit.

The earphone may include an ear plug having a terminal to be connected to an earphone connecting jack of the user terminal and a switching unit configured to selectively connect a third pole of the terminal of the earphone plug to the measuring unit.

The sensor driving signal may be transferred through a first pole of the terminal of the earphone, and when the sensor driving signal is transferred, a third pole of the terminal of the earphone may be connected to the measuring unit.

The sensor driving signal may be applied to the switching unit through the first pole. The switching unit may include a filter unit configured to pass a sensor driving signal having a frequency outside the predetermined frequency range, and a switch configured to connect the third pole to the measuring unit when applied with the sensor driving signal passed through the filter unit.

The switching unit may further include a rectifying unit positioned between the filter unit and the switch to rectify the sensor driving signal passed through the filter unit into direct current (DC).

The filter unit may be a low-pass filter passing only a signal smaller than the first frequency or a high-pass filter passing only a signal larger than the second frequency.

The switch may connect the third pole to the measuring unit when a direct current voltage equal to or greater than a predetermined intensity is inputted from the rectifying unit.

According to the present disclosure, it is possible to naturally measure the user's body temperature and other biosignals while the user is wearing earphone on his or her ears and using a smartphone. In particular, by taking measurement during a mute interval, it is possible to prevent inaccurate measurements from being taken due to vibration or sound that may be generated in the process of converting an audio electric signal outputted from the earphone into sound and outputting the result. In addition, the problem of delay in measuring, which may occur according to characteristics of the sensor, can be solved, so that user inconvenience can be prevented.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a block diagram illustrating a configuration of a biosignal measuring system using an earphone according to an embodiment of the present disclosure;

FIG. 2 is an audio waveform diagram provided to explain a mute interval according to the present disclosure;

FIG. 3 is a block diagram showing a configuration of a biosignal measuring system using an earphone according to another embodiment of the present disclosure;

FIG. 4 is a view illustrating a right earbud of an earphone according to an embodiment of the present disclosure;

FIG. 5 is a flowchart provided to explain an operation of a biosignal measuring system using an earphone according to an embodiment of the present disclosure;

FIG. 6 is a flowchart provided to explain an operation of a biosignal measuring system using an earphone according to another embodiment of the present disclosure; and

FIGS. 7 and 8 are graphs showing changes in sensor temperature measured after earphone are worn according to an embodiment of the present disclosure.

### DETAILED DESCRIPTION

Hereinafter, preferred embodiments of the present disclosure will be described in detail with reference to the accompanying drawings so that those with ordinary knowledge in the art may easily achieve the present disclosure.

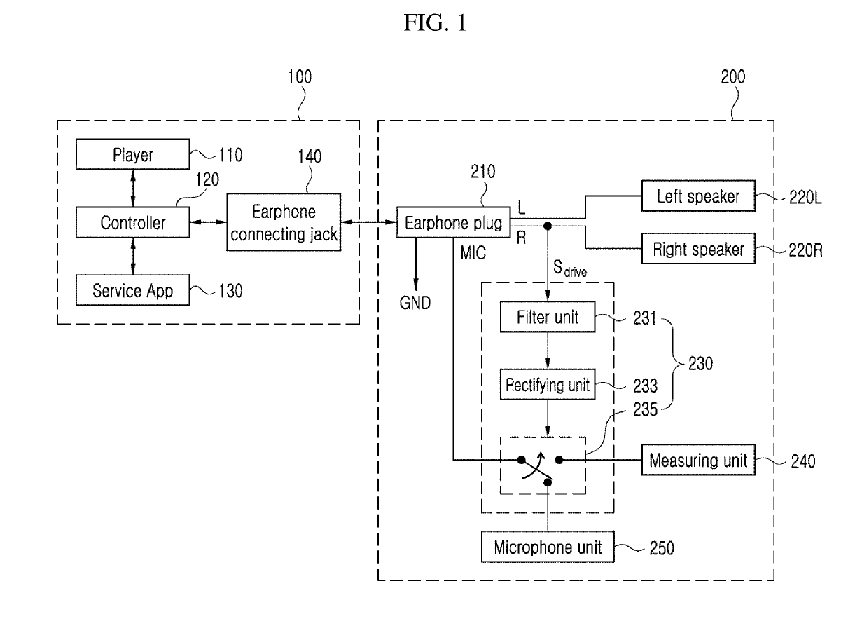

FIG. 1 is a block diagram illustrating a configuration of a biosignal measuring system using an earphone according to an embodiment of the present disclosure.

Referring to FIG. 1, the biosignal measuring system using an earphone according to the present disclosure may include a user terminal 100 and earphone 200.

The user terminal 100 may be realized as an information communication terminal such as a smart phone, a tablet PC, a personal digital assistant (PDA), a web pad and so on, which is provided with a memory unit and also equipped with a microprocessor for computation capability.

The user terminal 100 may play content such as music, video and so on that includes sound information. The user terminal 100 may output the sound information contained in the content through a speaker (not shown) provided therein or may transfer the sound in the form of an audio electric signal to the earphone 200 so that the sound is outputted.

The user terminal 100 may measure the user's biosignal through the earphone 200. The user terminal 100 may receive the measured biosignal information from the earphone 200 and store the biosignal information in a memory (not shown). The user terminal 100 may display the biosignal information transferred from the earphone 200 on a screen.

The user terminal 100 may provide the biosignal information measured at the earphone 200 to a service server (not shown) through a communication network.

The communication network may include a variety of data communication networks including Local Area Network (LAN), Metropolitan Area Network (MAN), Wide Area Network (WAN), Internet, 3rd Generation (3G), 4th Generation (4G), Wi-Fi, Wireless Broadband Internet (WIBRO), or Long Term Evolution (LTE), whether wired or wireless, and using any communication method.

The biosignal measured at the earphone 200 may be a body temperature, an electrocardiogram (ECG), an electroencephalogram (EEG), an electromyogram (EMG), an electrooculogram (EOG), pulse rate and so on.

Hereinafter, an example in which the earphone 200 measures the body temperature will be described.

The user terminal 100 may include a player 110, a controller 120, a service application 130, an earphone connecting jack 140, and so on. Although not shown in FIG. 1, the user terminal 100 may also include a display module to visually output information, a speaker module to output information by sound, a memory module to store data and programs, a button or a touch panel to receive a user command, a communication module for communication with an external device, a battery module to supply operation power, an inertial sensor, a camera module and so on.

The player 110 performs a function of playing content including sound information such as music, video, etc., on the user terminal 100. The player 110 may be an audio playing application, a video playing application and so on.

The controller 120 may include a Central Processing Unit (CPU) and an operating system, and controls the overall operation of the user terminal 100. For example, Android smartphones may include CPU and Android platforms, and iPhone smartphones may include CPU and iOS.

The controller 120 may output an audio electric signal corresponding to the sound portion of the content played by the player 110 to the earphone 200 through the earphone connecting jack 140.

The controller 120 may monitor whether a mute interval occurs during playing of the content by the player 110, and may notify the service application 130 of it. Of course, the mute interval information may be directly transferred from the player 110 to the service application 130 instead of being transferred via the controller 120. The mute interval information may be information on the start or end of the mute interval.

When the player 110 continuously plays the songs included in the music playlist, there may generally be a mute interval between the songs in which no sound is outputted.

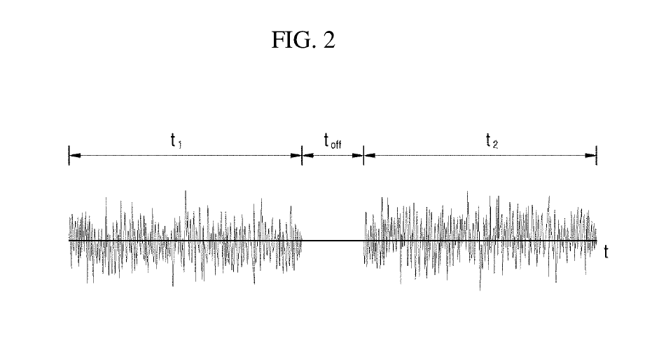

FIG. 2 is an audio waveform diagram provided to explain a mute interval according to the present disclosure.

As illustrated in FIG. 2, sound may be outputted while the first content is played for time t₁. And, there may be a mute interval in which no sound is outputted for a predetermined time t_{off}. Then sound may be outputted while the second content is played for time t₂.

Meanwhile, a mute interval may naturally occur between songs, but the mute interval may also be forcibly generated to measure a biosignal. For example, it is also possible to forcibly stop the operation of the player 110 from playing the content to generate a mute interval. Alternatively, while the player 110 is operated normally, in order to generate the mute interval, the output of the audio electric signal to the earphone 200 may be blocked. Accordingly, all the mute intervals may include the interval during which the audio electric signal is not transferred from the user terminal 100 to the earphone 200.

To measure the biosignal such as body temperature that can be measured in a relatively short time, the mute interval between songs may be utilized, while the mute interval may be forcibly generated for certain signals such as electrocardiogram that requires several tens of seconds or longer time for measurement.

Referring again to FIG. 1, the service application 130 may drive the measuring unit 240 provided in the earphone 200 to measure the body temperature. The service application 130 may receive the body temperature information measured at the earphone 200 to display the body temperature information on the screen of the user terminal 100 or store the body temperature information in a memory. The service application 130 may provide the body temperature information measured at the earphone 200 to the service server.

The service application 130 may be running in the background of the user terminal 100, measuring and collecting the body temperature at every predetermined time point through the earphone 200 without the user recognizing it. Of course, the body temperature measured at the earphone 200 may be received at a requested time point and displayed on the screen, in response to a request by a user for body temperature measurement that is made on a menu provided by the service application 130.

While the player 110 is continuously playing a plurality of songs according to the playlist, the service application 130 may transfer a sensor driving signal to the earphone 200 to cause the earphone 200 to measure the body temperature during the mute interval.

The earphone connecting jack 140 is connected to the earphone plug 210 to allow various signals to be exchanged between the user terminal 100 and the earphone 200.

The earphone connecting jack 140 may be realized as a 3.5 mm 4-pole terminal. Of course, depending on embodiments, the earphone connecting jack 140 may be a 2-pole terminal or a 3-pole terminal instead of a 4-pole terminal, or may have other types of terminals. Hereinafter, the embodiment of the present disclosure will be described with reference to an example of the 4-pole terminal.

The 4-pole terminal of the earphone connecting jack 140 may include a left audio signal terminal L, a right audio signal terminal R, a ground GND, and a microphone signal terminal MIC. The order of these terminals in the 4-pole terminal may vary between USA and European styles. The US style has an order of the left audio signal terminal L, the right audio signal terminal R, the ground GND, and the microphone signal terminal MIC. The European style has an order of the left audio signal terminal L, the right audio signal terminal R, the microphone signal terminal MIC, and the ground GND.

The user terminal 100 may transfer a left audio signal and a right audio signal to the earphone 200 through the left audio signal terminal L and the right audio signal terminal R. The user terminal 100 may receive the audio electric signal outputted from the microphone unit 250 from the earphone 200 through the microphone signal terminal MIC. Further, when there is a volume adjust button and so on in the earphone 200, a control signal corresponding to manipulation of the corresponding button may also be transferred.

The user terminal 100 may transfer a sensor driving signal S_{drive} through the left audio signal terminal L or the right audio signal terminal R. FIG. 1 illustrates an example in which the sensor driving signal S_{drive} is transferred through the right audio signal terminal R.

The sensor driving signal S_{drive} is preferably a signal having a voltage of a predetermined intensity that has a frequency outside the human-audible frequency range. As a result, the sensor driving signal S_{drive} transferred to the left audio signal terminal L or the right audio signal terminal R may be prevented from being heard by the user through the left speaker 220L or the right speaker 220R. Specifically, the audio electric signal has a frequency corresponding to a frequency range of 20 Hz to 20,000 Hz, which is audible frequency range, and the sensor driving signal S_{drive} may be realized to have a frequency corresponding to a frequency band below 20 Hz or above 20,000 Hz. Of course, depending on the characteristics of the voice unit of the left speaker 220L or the right speaker 220R, the audio electric signal may be outputted as a sound in a narrower range than 20 Hz to 20,000 Hz. For example, the audio electric signal may be outputted as a sound only in a range of 40 Hz to 15,000 Hz. Thus, depending on embodiments, the frequency range of the audio electric signal and the frequency range of the sensor driving signal S_{drive} may vary.

Meanwhile, the sensor driving signal S_{drive} transferred to the left audio signal terminal L or the right audio signal terminal R at the vicinity of the boundary of the audible frequency band may have an effect such that it is heard by the user through the left speaker 220L or the right speaker 220R. In order to solve such problem, a filter (not shown) for filtering signals outside the audible frequency range (e.g., a band pass filter that passes only the signals in the audible frequency range) may be provided at the front end of the left speaker 220L or the right speaker 220R.

The earphone 200 may include an earphone plug 210, a left speaker 220L, a right speaker 220R, a switching unit 230, a measuring unit 240, and a microphone unit 250.

The earphone plug 210 has a structure corresponding to the earphone connecting jack 140. For example, when the earphone connecting jack 140 has an US version 4-pole terminal structure, the earphone plug 210 may also have an US version 4-pole terminal structure.

The first pole L of the earphone plug 210 may transfer the left audio electric signal transferred from the user terminal 100 to the left speaker 220L. The second pole R of the earphone plug 210 may transfer the right audio electric signal transferred from the user terminal 100 to the right speaker 220R. The third pole MIC of the earphone plug 210 may be selectively connected to the measuring unit 240 and the microphone unit 250 through the switching unit 230. In addition, the fourth pole GND of the earphone plug 210 is the ground.

The switching unit 230 is configured to selectively connect the measuring unit 240 or the microphone unit 250 to the third pole MIC.

The switching unit 230 may normally connect the microphone unit 250 and the third pole MIC, and then connect the third pole MIC and the measuring unit 240 when the sensor driving signal S_{drive} is transferred from the user terminal 100.

When the sensor driving signal S_{drive} is inputted through the left audio signal terminal L or the right audio signal terminal R, the switching unit 230 may disconnect the third pole MIC from the microphone unit 250 to connect to the measurement unit 240. When the sensor driving signal is no longer applied, the switching unit 230 may re-connect the third pole MIC to the microphone unit 250.

The switching unit 230 may include a filter unit 231, a rectifying unit 233 and a switch 235 so as to process the sensor driving signal S_{drive} having a frequency outside the audible frequency range.

The filter unit 231 is configured to pass a sensor driving signal S having a frequency outside the audible frequency range, while blocking the audio electric signal having a frequency corresponding to a frequency band of 20 Hz to 20,000 Hz (i.e., audible frequency range). To this end, the filter unit 231 may be realized as a high-pass filter that passes only a signal having a frequency above a predetermined frequency or a low-pass filter that passes only a signal having a frequency below a predetermined frequency. For example, the filter unit 231 may be realized as a high-pass filter that passes only signals above 20,000 Hz, or a low-pass filter that passes only signals below 20 Hz. That is, the filter unit 231 may be realized with the high-pass filter when the sensor driving signal S has a frequency above the audible frequency range, while the filter unit 231 may be realized with the low-pass filter when the sensor driving signal S_{drive} has a frequency below the audible frequency range. With the filter unit 231, it is possible to prevent the audio electric signal from being applied to the switch 235 to cause malfunction.

The rectifying unit 233 is positioned between the filter unit 231 and the switch 235 to rectify the sensor driving signal S_{drive} that has passed through the filter unit 231 into direct current and output the direct current.

The switch 235 may be realized as a switching device that disconnects the third pole MIC from the microphone unit 250 to connect to the measuring unit 240 when a direct current voltage of a predetermined intensity or greater is inputted.

According to an embodiment, the switching unit 230 may also be realized such that, when the sensor driving signal is applied in the form of a pulse, the switching unit 230 connects the third pole MIC to the measuring unit 240 and automatically re-connects to the microphone unit 250 after a predetermined time has elapsed.

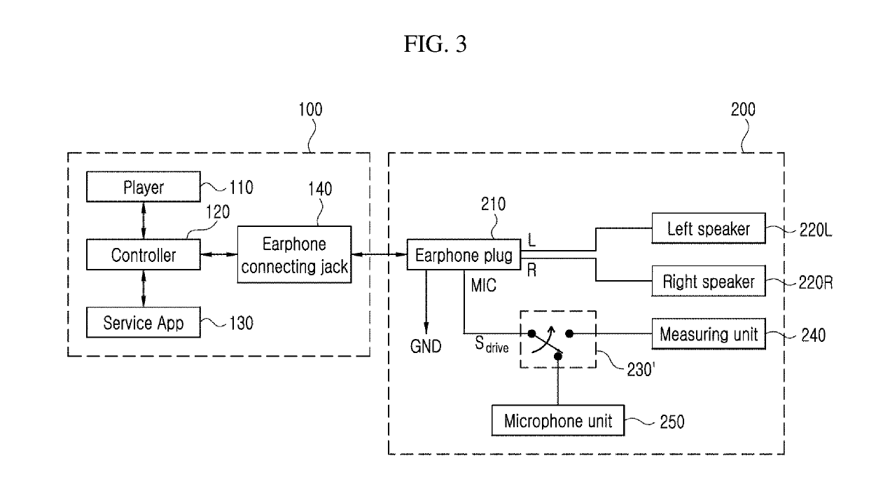

FIG. 3 is a block diagram showing a configuration of a biosignal measuring system using an earphone according to another embodiment of the present disclosure.

As shown in FIG. 3, a biosignal measuring system is different from the system shown in FIG. 1 in that the sensor driving signal S_{drive} is transferred to the switching unit 230′ through the microphone signal terminal MIC.

In the embodiment shown in FIG. 3, since the sensor driving signal S_{drive} is transferred through the microphone signal terminal MIC, the switching unit 230′ does not include the filter unit 231 and the rectifying unit 233, and may only include the switching device to disconnect the third pole MIC from the microphone unit 250 to connect to the measuring unit 240 when the input signal S is inputted.

Except for the switching unit 230′, the other components of the biosignal measuring system illustrated in FIG. 3 may operate in the same manner as the corresponding components having the same reference numerals in the system of FIG. 1.

Meanwhile, according to an embodiment, unlike FIGS. 1 and 3, the switching unit may be realized as a toggle switch and so on. The user may selectively connect the third pole MIC to the microphone unit 250 or to the measuring unit 240 by manually operating the switching unit. The switching unit may be mounted on an earphone remote control (not shown) that may provide a volume control function and so on. When the user connects the measuring unit 240 to the third pole MIC by operating the toggle switch, the measuring unit 240 may measure the biosignal and transfer the measured biosignal to the user terminal 100 through the third pole MIC. The service application 130 may process the biosignal inputted through the third pole MIC to display on the screen or store in the memory.

The measuring unit 240 may be configured to measure a user's biosignal, and may include a sensor and a sensor circuit for this purpose. For example, when the biosignal of the measured subject is a body temperature, the measuring unit 240 may include a thermistor having electrical resistance value varying according to temperature. Of course, the measuring unit 240 may also use a sensor other than a thermistor to measure the body temperature.

Meanwhile, when the biosignal of the measured subject is an electrocardiogram, an electroencephalogram, an electromyogram, an electrooculogram, a pulse rate, etc., the measuring unit 240 may include another sensor suitable for such biosignal of the measured subject.

Further, while FIGS. 1 and 3 show one measurement unit 240 by way of example, it is also possible to include a plurality of measuring units 240. The sensor driving signal may also be distinguished and transferred according to a sensor to be driven. For example, the body temperature sensor driving signal and the electrocardiograph sensor driving signal may be distinguished from each other.

Meanwhile, the measuring unit 240 may be provided on both the left earbud and the right earbud of the earphone 200, or may be provided on only one of the left and right earbuds in view of cost and so on.

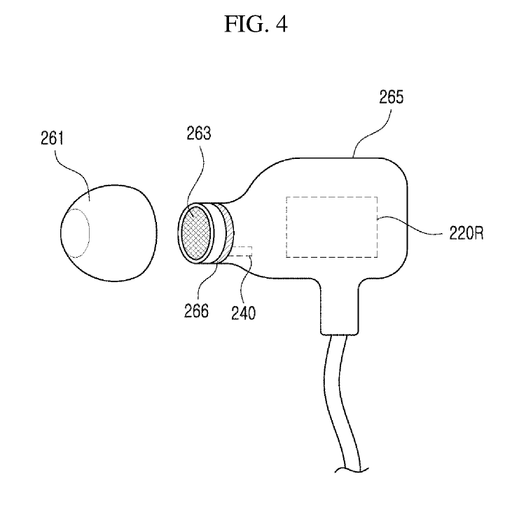

FIG. 4 is a view illustrating a right-side earbud of an earphone according to an embodiment of the present disclosure.

Referring to FIG. 4, the right earbud may include an earpiece 261, a screen 263, a housing 265, a right speaker 220R, a measuring unit 240 and so on.

The housing 265 may have the right speaker 220R and the measuring unit 240 mounted therein. In addition, the sound outputted from the right speaker 220R may be outputted to the outside through the screen 263. In FIG. 4, the position where the right speaker 220R and the measuring unit 240 are mounted in the housing 265 is only an example, and the mounting position may vary according to embodiments.

The earpiece 261 may be mounted on one end of the housing 265 and inserted into the user's ear canal. The earpiece 261 may be made of an elastic material such as polysilicon, polyurethane, rubber, latex and so on so that it may be brought into close contact with the ear canal of a user when inserted. Particularly, the earpiece 261 may be made of a material such as polysilicon, polyurethane, rubber, latex and so on mixed or coated with a material having good thermal conductivity or electrical conductivity such that, when the earphone is inserted into the ear canal of the user, the biosignal is transferred to the measuring unit 240 efficiently.

A connecting member 266 may be formed on a portion of the housing 265. The connecting member 266 may be realized with a metal material or an elastic material having good thermal conductivity or electrical conductivity to electrically or thermally connect the earpiece 261 and the measuring unit 240. For example, as illustrated in FIG. 4, at least a portion of the connecting member 266 is exposed along the outer periphery of the housing 265 to contact the ear piece 261, while at least a portion of the connecting member 266 is exposed through the inner surface of the housing 265 to contact the measuring unit 240. As a result, the connecting member 266 may electrically or thermally connect the ear piece 261 mounted on one end of the housing 265 to the measuring unit 240 mounted inside the housing 265.

The earphone 200 may selectively include a microphone unit 250. For example, when the earphone 200 does not provide a microphone function, the earphone 200 may not include the microphone unit 250. Meanwhile, when the earphone 200 provides the volume control function, a volume control button (not shown) may be connected to the third pole MIC.

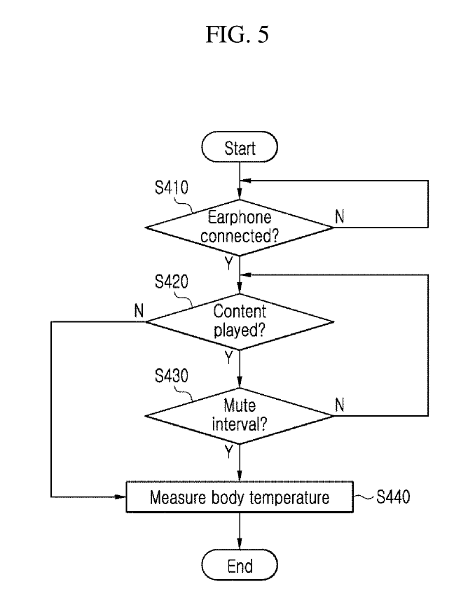

FIG. 5 is a flowchart provided to explain an operation of a biosignal measuring system using an earphone according to an embodiment of the present disclosure.

Referring to FIGS. 1 and 5, when the earphone 200 is connected to the user terminal 100, at S410-Y, the service application 130 may determine whether the player 110 is playing the content, at S420. The service application 130 may request the player 110 to determine whether the content is played or not, and receive the result. Alternatively, the service application 130 may determine whether the player 110 is playing the content through the controller 120.

When the player 110 is not playing the content at S420-N, the service application 130 may transfer a sensor driving signal directly to the earphone 200 to cause the body temperature measurement to be performed, at S440.

Meanwhile, when the player 110 is playing the content at S420-Y, the service application 130 may monitor whether it 110 enters into the mute interval, at S430. In one implementation, the service application 130 may be provided with a message from the player 110 indicating about the mute interval. Alternatively, the service application 130 may determine it through the controller 120.

When determining the mute interval at S430-Y, the service application 130 may transfer a sensor driving signal to the earphone 200 such that the body temperature may be measured at S440.

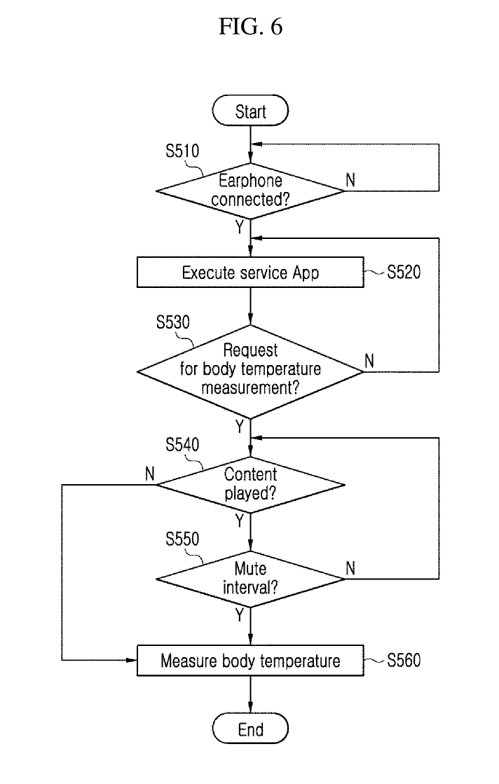

FIG. 6 is a flowchart provided to explain an operation of a biosignal measuring system using an earphone according to another embodiment of the present disclosure.

Referring to FIG. 6, when the earphone 200 is connected to the user terminal 100 at S510-Y, the service application 130 may be automatically executed at S520.

Next, when the user requests the body temperature measurement through the service application 130 at S530-Y, the service application 130 may determine whether the player 110 is playing the content at S540. The service application 130 may request the player 110 to determine whether the content is played or not, and receive the result. Alternatively, the service application 130 may determine whether the player 110 is playing the content through the controller 120.

When the player 110 is not playing the content at S540-N, the service application 130 may transfer a sensor driving signal directly to the earphone 200 to cause the body temperature measurement to be performed, at S560.

Meanwhile, when the player 110 is playing the content at S540-Y, the service application 130 may monitor whether it 110 enters into the mute interval, at S550. In one implementation, the service application 130 may be provided with a message from the player 110 indicating about the mute interval. Alternatively, the service application 130 may determine it through the controller 120.

When determining the mute interval at S550-Y, the service application 130 may transfer a sensor driving signal to the earphone 200 such that the body temperature may be measured at S560.

Meanwhile, there may be a difference between the body temperature of the user and the temperature measured at the measuring unit 240 (hereinafter referred to as ‘sensor temperature’) depending on the time point the body temperature is measured. For example, after a user inserts an earbud with a thermistor mounted therein into his or her ear, a delay occurs because it takes a certain amount of time before the resistance value of the thermistor changes to match the body temperature.

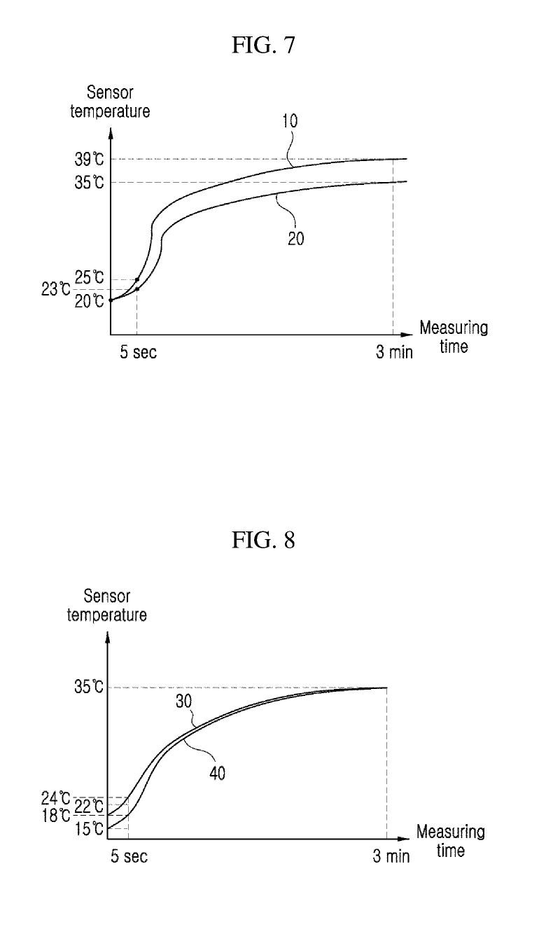

FIGS. 7 and 8 are graphs showing changes in sensor temperature measured after earphone are worn according to an embodiment of the present disclosure.

In FIG. 7, the graph 10 shows that the sensor temperature is 20° C. when the measurement is started, and then rises to 25° C. past 5 seconds and finally converges to 39° C. The graph 20 shows that the sensor temperature is 20° C. when the measurement is started, and then rises to 23° C. past 5 seconds, and finally converges to 35° C.

In FIG. 8, the graph 30 shows that the sensor temperature is 18° C. when the measurement is started, and then rises to 22° C. past 5 seconds and finally converges to 35° C. The graph 40 shows that the sensor temperature is 15° C. when the measurement is started, and then rises to 22° C. past 5 seconds, and finally converges to 35° C.

As shown in FIGS. 7 and 8, it can be seen that a certain delay time is required until the temperature measured at the measuring unit 240 reaches the user's body temperature. In the embodiments of FIGS. 7 and 8, a delay time of about 3 minutes is required. But it may considerably inconvenience the user if it takes 3 minutes to obtain the body temperature measurement result after he or she wears the earphone 200.

In order to solve this problem, when the time of connection between the earphone 200 and the user terminal 100 is less than a predetermined reference value so that it is before when the sensor temperature reaches the body temperature, the user terminal 100 may provide a predicted body temperature to the user based on the sensor temperature change rate. To this end, the user terminal 100 may store in advance the experiment data of analyzing the correlativity between sensor temperature change rate and body temperature in the memory in the form of a table or a graph. Alternatively, the user terminal 100 may be provided with, or refer to the experimental data of analyzing the correlativity between the change rate of the sensor temperature and the body temperature from the outside through a communications network. Various experimental data may be prepared in advance according to various criteria such as gender, age, season, time and so on.

Of course, it is also possible to predict the body temperature using the following equation:

$${T}_{e} = A \frac{( {T}_{2} - {T}_{1} )}{( {t}_{2} - {t}_{1} )} + B \qquad [ Equation 1 ]$$

where, Tₑ is the predicted body temperature value, T₁ is the sensor temperature measured at time t₁, T₂ is the sensor temperature measured at time t₂, and A and B are parameters determined according to the characteristics of the measuring unit 240 mounted in the earphone 200. Of course, Equation 1 is merely an example, and it is also possible to obtain and use another mathematical equation capable of predicting the body temperature more accurately.

In accordance with an embodiment, while displaying the predicted body temperature on the screen, the user terminal 100 may concurrently display a statement such as “This is predicted body temperature. The accurate body temperature will be provided again in x minutes” and the like.

Embodiments of the present disclosure include a computer-readable medium including program instructions for performing various computer-implemented operations. The medium records a program for executing the method described above. The medium may include program instructions, data files, data structures, etc., alone or in combination. Examples of such media include hardware devices, and so on configured to store and execute program instructions such as magnetic media such as hard disks, floppy disks and magnetic tape, optical recording media such as CD and DVD, floptical disk and magneto-optical media, ROM, RAM, flash memory, etc. Examples of the program instructions include machine language codes such as those generated by a compiler, as well as high-level language codes that may be executed by a computer using an interpreter, and so on.

The present disclosure has been described in detail. However, it should be understood that the detailed description and specific examples, while indicating preferred embodiments of the disclosure, are given by way of illustration only, since various changes and modifications within the scope of the disclosure will become apparent to those skilled in the art from this detailed description.

What is claimed is:

1. A biosignal measuring method using an earphone, comprising:
sensing the earphone being connected to a user terminal, wherein the earphone comprises a measuring unit for measuring a biosignal;
transferring a sensor driving signal from the user terminal to the connected earphone during a predetermined interval; and
driving the measurement unit according to the sensor drive signal to measure the biosignal.

2. The biosignal measuring method of claim 1, wherein the predetermined interval is a mute interval during which an audio electric signal is not transferred from the user terminal.

3. The biosignal measuring method of claim 2, wherein the mute interval is defined as an interval spanning from an end of playing a first content to a start of playing a second content in the user terminal.

4. The biosignal measuring method of claim 2, wherein the sensor driving signal is transferred through a first pole of terminals of the earphone, and when the sensor driving signal is transferred, the measuring unit is connected to a third pole of the terminals of the earphone.

5. The biosignal measuring method of claim 4, wherein the sensor driving signal has a frequency outside audible frequency range.

6. The biosignal measuring method of claim 5, wherein the sensor driving signal has a voltage equal to or greater than a predetermined intensity.

7. The biosignal measuring method of claim 4, wherein the sensor driving signal has a frequency outside a predetermined frequency range,
the predetermined frequency range is equal to or greater than a first frequency and equal to or less than a second frequency, and
the sensor driving signal has a voltage equal to or greater than a predetermined intensity.

8. The biosignal measuring method of claim 1, wherein the biosignal is at least one of body temperature, electrocardiogram (ECG), electroencephalogram (EEG), electrocardiogram (EMG), electrooculogram (EOG), and pulse rate.

9. The biosignal measuring method of claim 1, wherein the biosignal is body temperature, and when a duration in which the earphone is connected to the user terminal is equal to or less than a predetermined reference value, the body temperature of the user, which is predicted based on a rate of change of the temperature measured at the measuring unit, is displayed.

10. A biosignal measuring system using an earphone, comprising:
an earphone comprising a measuring unit for measuring a biosignal; and
a user terminal configured to transfer a sensor driving signal to drive the measuring unit during a mute interval in which an audio electric signal is not outputted to the earphone, and receive measured biosignal from the measuring unit.

11. The biosignal measuring system of claim 10, wherein the mute interval is defined as an interval spanning from an end of playing a first content to a start of playing a second content in the user terminal.

12. The biosignal measuring system of claim 10, wherein the biosignal is body temperature, and when a duration in which the earphone is connected to the user terminal is equal to or less than a predetermined reference value, the user terminal displays the body temperature of the user which is predicted based on a rate of change of the temperature measured at the measuring unit.

13. The biosignal measuring system of claim 10, wherein the earphone further comprises:
an ear plug having a terminal to be connected to an earphone connecting jack of the user terminal; and
a switching unit configured to selectively connect a third pole of the terminal of the earphone plug to the measuring unit,
wherein the sensor driving signal is transferred through a first pole of the terminal of the earphone, and
when the sensor driving signal is transferred, the third pole of the terminal of the earphone is connected to the measuring unit.

14. The biosignal measuring system of claim 13, wherein the sensor driving signal has a frequency outside audible frequency range, and is applied to the switching unit through the first pole.

15. The biosignal measuring system of claim 13, wherein the sensor driving signal has a voltage equal to or greater than a predetermined intensity.

16. The biosignal measuring system of claim 15, wherein the sensor driving signal has a frequency outside a predetermined frequency range, and
the switching unit comprises:
a filter unit configured to pass a sensor driving signal having a frequency outside the predetermined frequency range; and
a switch configured to connect the third pole to the measuring unit when applied with the sensor driving signal passed through the filter unit.

17. The biosignal measuring system of claim 16, wherein the switching unit further comprises a rectifying unit positioned between the filter unit and the switch to rectify the sensor driving signal passed through the filter unit into direct current (DC).

18. The biosignal measuring system of claim 16, wherein the predetermined frequency range is equal to or greater than a first frequency and equal to or less than a second frequency, and
the filter unit is a low-pass filter passing only a signal smaller than the first frequency or a high-pass filter passing only a signal larger than the second frequency.

19. The biosignal measuring system of claim 16, wherein the switch connects the third pole to the measuring unit when a direct current voltage equal to or greater than a predetermined intensity is inputted from the rectifying unit.

20. The biosignal measuring system of claim 10, wherein the biosignal is at least one of body temperature, electrocardiogram (ECG), electroencephalogram (EEG), electrocardiogram (EMG), electrooculogram (EOG), and pulse rate.
